(19) United States
(12) Patent Application Publication
(10) Pub. No.: US 2015/0027484 A1
(43) Pub. Date: Jan. 29, 2015

(54) LIQUID PIGMENT COMPOSITION IN AN APPLICATOR PEN TO PROVIDE INSTANT DRYING ON A HAIR STRAND AND TEMPORARY HAIR COLORATION UNTIL A FIRST WASH

(71) Applicant: Maura Keenan Narbut, Atherton, CA (US)

(72) Inventor: Maura Keenan Narbut, Atherton, CA (US)

(21) Appl. No.: 14/445,317

(22) Filed: Jul. 29, 2014

Related U.S. Application Data
(60) Provisional application No. 61/859,303, filed on Jul. 29, 2013.

(51) Int. Cl. A45D 19/00; A61Q 5/06; A61K 8/19; A45D 24/02; A45D 34/04

(52) U.S. Cl. CPC A45D 19/0008; A45D 24/02; A45D 34/04; A61K 8/19; A61Q 5/065; A45D 2019/0083
USPC 132/208; 132/313; 132/221

(57) ABSTRACT

Disclosed are a composition, method, a device and a system of an applicator pen having a unique liquid pigment to provide instant drying on a hair strand and temporary hair coloration until a first wash. In one embodiment, a hair coloring kit includes a liquid pigment having a first property that enables the liquid pigment to dry upon contact with a hair surface, and which exhibits a second property through which the liquid pigment is removable from the hair surface upon a first wash with a shampoo material, and an applicator pen to apply the liquid pigment to the hair surface through a capillary action through which the liquid pigment is extruded through the applicator pen onto the hair surface. The liquid pigment is removable from a skin adjacent to the hair surface when washed using the shampoo material.

### CLAIM OF PRIORITY

This application claims priority from U.S. Provisional Application No. 61/859,303 titled “COMPOSITION, METHODS AND KIT FOR COLORING HAIR ROOTS” filed on Jul. 29, 2013.

### FIELD OF THE TECHNOLOGY

This disclosure relates generally to the field of hair dyes and, more particularly, to an applicator pen having a unique liquid pigment composition to provide instant drying on a hair strand and temporary hair coloration until a first wash.

### BACKGROUND

A pigment (temporary hair color) may available in powder and/or a viscous form such as in rinses, shampoos, gels, sprays, and/or foams. Temporary hair color may be brighter and more vibrant than semi-permanent and permanent hair color. It may be used to color hair for special occasions (e.g., costume parties, Halloween, birthday parties, etc.). The pigment molecules in temporary hair color may be large and may not be able to penetrate the cuticle layer. The color particles may remain adsorbed (closely adherent) to the hair shaft and may be removed with a single shampooing. Temporary hair color may persist on hair that is excessively dry or damaged in a way that allows for migration of the pigment to the interior of the hair shaft. However, the pigment may be waxy, so that it must first be washed off before a long term hair color is applied. In addition the pigment may not be suitable for application through an applicator pen because of its powder and/or viscous form. Therefore it may be difficult to apply the pigment evenly on human hair.

### SUMMARY

Disclosed are a composition, method, a device and a system of an applicator pen having a unique liquid pigment composition to provide instant drying on a hair strand and temporary hair coloration until a first wash.

In one aspect, a hair coloring kit comprising a liquid pigment having a first property that enables the liquid pigment to dry upon contact with a hair surface, and which exhibits a second property through which the liquid pigment is removable from the hair surface upon a first wash with a shampoo material, and an applicator pen to apply the liquid pigment to the hair surface through a capillary action through which the liquid pigment is extruded through the applicator pen onto the hair surface. The liquid pigment is removable from a skin adjacent to the hair surface when washed using the shampoo material. The liquid pigment includes an inorganic pigment comprising at least one iron oxide in a concentration between 1.5% weight to 12.5% weight. The liquid pigment is substantially free of ethyl alcohol. A viscosity of the liquid pigment is less than 100 centipoise (cP) to prevent clogging of a chamber of the applicator pen. The hair surface is optionally treatable with a dye composition while the liquid pigment is on the hair surface and without requiring a wax removal process. Further, a comb is usable with the applicator pen to expose a hair root while shielding a skin that underlies the hair surface.

The hair coloring kit may include a reservoir member in a central chamber of the applicator pen that stores the liquid pigment. The reservoir member may be removable from the applicator pen when a back-end assembly is unfastened from an end opposite to an applicator tip of the applicator pen such that the back-end assembly to expose the reservoir member and to permit the reservoir member to slide outward from a cavity of the central chamber of the applicator pen in the end opposite to the applicator tip.

The hair coloring kit may include a removable cap on secure the applicator tip of the applicator pen and to prevent the liquid pigment from at least one of drying and escaping from the applicator pen when the removable cap is secured to the applicator pen. The liquid pigment may automatically extrude from the applicator tip of the applicator pen at a rate of volume that does not create an excess leakage of the liquid pigment onto a surrounding skin. The liquid pigment extruding from the applicator tip may be substantially saturated in the hair surface based on a regulated rate of extrusion of the liquid pigment. A wicking action of the applicator pen may permit the liquid pigment from extruding from the applicator pen through the applicator tip onto the hair surface. The liquid pigment may include a water, a water soluble film forming polymer, a surfactant, and/or an inorganic pigment. The water soluble film forming polymer may a polyacrylic acid, an acrylate/octylacrylamide copolymer, and/or a polyvinylpyrrolidone polymer. A body of the applicator pen may be at least one of a transparent visibility and an opaque visibility to permit a level of the liquid pigment to be visually observable to a user of the applicator pen.

In another aspect, a hair coloring kit includes a liquid pigment having a first property that enables the liquid pigment to dry upon contact with a hair surface, and which exhibits a second property through which the liquid pigment washes out upon a wash with a shampoo material, and an applicator pen to apply the liquid pigment to the hair surface through a capillary action through which the liquid pigment is extruded through the applicator pen onto the hair surface. The liquid pigment is removable from a skin adjacent to the hair surface when washed using a shampoo material. The liquid pigment includes an inorganic pigment comprising at least one iron oxide in a concentration between 1.5% weight to 12.5% weight. The liquid pigment is substantially free of ethyl alcohol. The hair surface is optionally treatable with a dye composition while the liquid pigment is on the hair surface and without requiring a wax removal process. A viscosity of the liquid pigment is less than 100 cP to prevent clogging of a chamber of the applicator pen.

In another aspect, a method includes applying a liquid pigment to a hair surface through a capillary action through which the liquid pigment is extruded through an applicator pen onto the hair surface. The liquid pigment is removable from a skin adjacent to the hair surface when washed using a shampoo material. The liquid pigment may include an inorganic pigment comprising at least one iron oxide in a concentration between 1.5% weight to 12.5% weight. The liquid pigment is substantially free of ethyl alcohol. A viscosity of the liquid pigment is less than 100 cP to prevent clogging of a chamber of the applicator pen. The method includes exposing a hair root while shielding a skin that underlies the hair surface using a comb utilized in conjunction with the applicator pen. The hair surface is optionally treatable with a dye composition while the liquid pigment is on the hair surface and without requiring a wax removal process. The liquid pigment has a property that enables the liquid pigment to dry upon contact with the hair surface and remain in liquid form while in the applicator pen.

The compositions, methods, and kits disclosed herein may be implemented in any means for achieving various aspects. Other features will be apparent from the accompanying drawings and from the detailed description that follows.

### BRIEF DESCRIPTION OF THE DRAWINGS

The embodiments of this invention are illustrated by way of example and not limitation in the figures of the accompanying drawings, in which like references indicate similar elements and in which:

FIG. 1 is a perspective view of an applicator capable of applying the hair dye composition, according to one or more embodiments.

FIG. 2 is a perspective view of the applicator of FIG. 1 with a cap removed displaying an applicator tip, according to one or more embodiments.

FIG. 3A is a perspective view of the applicator of FIG. 2 with a back end removed displaying a reservoir member partially removed, according to one or more embodiments.

FIG. 3B is a perspective view of the applicator of FIG. 2 with the reservoir member fully removed, according to one or more embodiments.

FIG. 4 is a perspective view of a comb that can abide in applying a hair dye composition, according to one or more embodiments.

FIG. 5 is a polymer view of water soluble film forming polymers, according to one or more embodiments.

Other features of the present embodiments will be apparent from the accompanying drawings and from the detailed description that follows.

### DETAILED DESCRIPTION

Disclosed are a composition, method, a device and a system of an applicator pen having a unique liquid pigment composition to provide instant drying on a hair strand and temporary hair coloration until a first wash. In the following description, for the purposes of explanation, numerous specific details are set forth in order to provide a thorough understanding of the various embodiments. It will be evident, however, to one skilled in the art that the various embodiments may be practiced without these specific details.

Example embodiments, as described below, may be used to provide a hair dye composition, kit, and method for coloring hair roots. Although the present embodiments have been described with reference to specific example embodiments, it will be evident that various modifications and changes may be made to these embodiments without departing from the broader spirit and scope of the various embodiments.

In one embodiment, the present disclosure provides a liquid pigment 301 (e.g., a type of hair dye composition for temporary coloring) for direct application to hair by capillary action (i.e. wicking). The liquid pigment 301 (e.g., a temporary liquid pigment 301) should therefore have a free-flowing viscosity (less than 100 cP) that allows for sufficient flow by capillary action. Further, the liquid pigment 301 should comprise inorganic pigment and be sufficiently free of ethyl alcohol. Still further, the liquid pigment 301 should be gentle and temporary or semi-permanent, allowing use on hair surfaces near to skin, and should be easily removable by washing with a detergent (e.g. shampoo).

Ethyl alcohol can have a drying effect that results in damage to hair (e.g. fizziness) and/or skin. In one or more embodiments, the liquid pigment 301 is sufficiently free of ethyl alcohol. In one or more embodiments, components of the liquid pigment 301 may be water soluble, minimizing the need for an organic solvent.

Hair dyes more permanent than the liquid pigment 301 disclosed herein often comprise organic pigments accompanied by oxidizing agents in an alkaline (i.e. high pH, basic) environment. Typically, oxidizing agents include hydrogen peroxide and the alkaline environment is provided by ammonia. In addition, many permanent and demi-permanent hair dyes comprise couplers (i.e. color developers), such as aniline derivatives, which may be toxic. Use of these chemicals can result in a range of adverse effects, including temporary skin irritation and allergy, hair breakage, skin discoloration and unexpected hair color results. Such hair dyes therefore need to be applied with great care to avoid contact with the skin. In one or more embodiments, the liquid pigment 301 disclosed herein may be a temporary hair dye or a semi-permanent hair dye. The liquid pigment 301 may be substantially free of organic pigments, oxidizing agents and couplers. The liquid pigment 301 may therefore be safe for direct use on areas of hair that are close to the skin, including hair roots, beard, mustache, eyebrows and/or eyelashes.

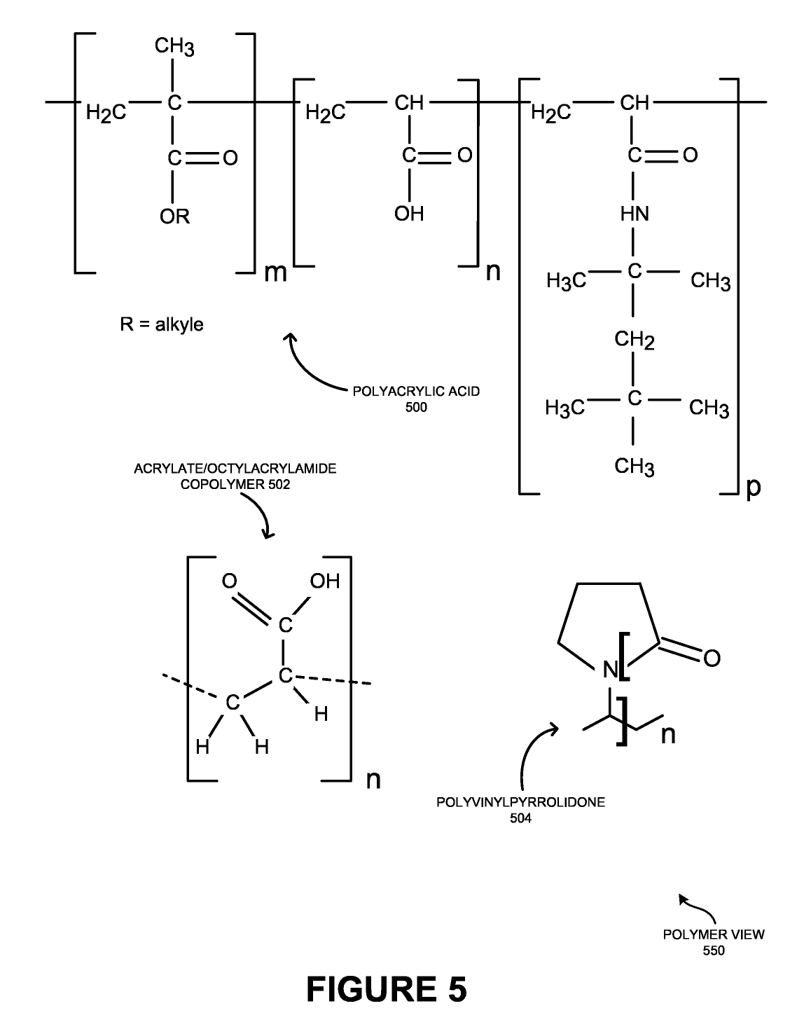

In one or more embodiments, the liquid pigment 301 may comprise water, one or more water soluble film forming polymers (also known as shielding, dye-shields, and anti-rub off), surfactants, and one or more inorganic pigments. See, for example, FIG. 5.

FIG. 5 is a polymer view 550, according to various embodiment. Particularly, FIG. 5 illustrates a polyacrylic acid polymer 500, an acrylate/octylacrylamide copolymer 502, and a polyvinylpyrrolidone 504. These are examples of water soluble film-forming polymers which may be found in the liquid pigment 301, in accordance with one embodiment. In another embodiment, the liquid pigment 301 may comprise these and other water soluble film-forming polymers familiar to one of ordinary skill in the art.

Upon application to hair, these polymers form a water resistant film on the hair. They may be used in varying concentrations. Polyacrylic acid 500 may be at a concentration at or between 0.05 percent weights (% wt) and 5% wt. An acrylate/octylacrylamide copolymer 502 may be at a concentration at or between 1% wt and 20% wt. Polyvinylpyrrolidone 504, also known as povidone, is not tacky and therefore does not interfere with the free capillary flow of the dye within the applicator. Polyvinylpyrrolidone 504 may be at a concentration at or between 1% wt and 20% wt.

In one or more embodiments, the liquid pigment 301 may comprise one or more surfactants. A surfactant is a compound that lowers the surface or interface tension between a liquid and a solid or another liquid. In one embodiment, the surfactants in the liquid pigment 301 may be sorbitan sesquioleates, propylene glycol, triethanolamine and/or other surfactants familiar to one of ordinary skill in the art. Sorbitan sesquioleates act as a moisturizer, and may be at a concentration at or between 1% wt. and 20% wt. Propylene glycol and Triethanolamine both act as emulsifiers, serving to stabilize the emulsion. Propylene glycol may be at a concentration at or between 1% wt and 20% wt. Triethanolamine may be at a concentration at or between 0.05% wt and 5% wt.

In one or more embodiments, the liquid pigment 301 should have a suitable acidity to allow the desired degree of permanence on the hair. A degree of permanence of a hair coloring may therefore be determined by adjusting the pH of the liquid pigment 301. The previously mentioned polyacrylic acid and acrylate/octylacrylamide copolymer are both acidic, while triethanolamine is basic. The ratio and strength of acidic and basic components of the liquid pigment 301 can together determine the pH and thereby determine the permanence of the hair coloring.

In one or more embodiments the one or more inorganic pigments may be one or more iron oxides at individual concentrations at or between 1.5% wt to 12.5% wt. Iron oxides are not irritating to skin, unlike many synthesized organic dyes and accompanying chemicals. As iron oxides come in a variety of colors, different liquid pigment 301s may comprise different combinations and/or concentrations of iron oxides.

Iron oxides may be solubilized at high temperatures and highly acidic aqueous conditions. Propylene glycol, previously described as a surfactant and emulsifier, may also act as a co-solvent that allows for iron oxide solubility at lower temperatures and less highly acidic conditions.

An example liquid pigment 301 that illustrates the present embodiment thereof is presented below. All concentrations are by percent weight unless otherwise indicated. Different iron oxides may provide a different color to the liquid pigment 301. The example below shows a black liquid pigment 301.

| Component | Percent Weight |
|---|---|
| Water | to 100 |
| Polyacrylic Acid | 0.4 |
| Acrylate/Octylacrylamide Copolymer | 8.9 |
| Polyvinylpyrrolidone | 9.5 |
| Sorbitan Sesquioleates | 2.1 |
| Propylene Glycol | 7.3 |
| Triethanolamine | 1.3 |
| Black Oxide of Iron | 1.5-2.4 |

In one aspect, the present embodiment provides a method of applying the previously described liquid pigment 301, through an applicator, to hair surfaces. A plurality of applicators are available to accommodate the hair type being colored and to aide in control of the flow of liquid pigment 301. The applicator in the present method is substantially a marker (alternatively known in the art as soft-tip pens, fiber-tip pens and felt-tip pens) configured to apply liquid pigment 301 to hair. The applicator is configured to wick (e.g. by capillary action) or otherwise transfer the liquid pigment 301 from a reservoir to a tip of the applicator. The advantage of using such an applicator is that dye does not drip from the applicator during use. The technical details of applicators and the manufacture thereof are well known. Applicators suitable for use according to the present embodiment are manufactured, for example, by such companies as Staedler Mars GmbH & Co. (Niirnberg, Germany), Faber-Castell Vertrieb GmbH (Stein, Germany), Dixon Ticonderoga Co. (Heathrow, Fla.), Pentel Co. Ltd. (Tokyo, Japan) and Pilot Corporation (Tokyo, Japan). The liquid pigment 301 should be of sufficiently low viscosity (less than 100 cP) and of such composition so as to flow through the applicator without clogging it and to flow easily from the applicator to the hair surfaces contacted thereby.

As discussed earlier, permanent and demi-permanent hair dyes may be toxic. Further, such hair dyes may be damaging to hair and skin when misapplied. The liquid pigment 301 discussed in this application is preferably a gentle and temporary or semi-permanent hair dye. In any of the embodiments of the method discussed below, the method may be performed by a non-hair professional without risk of damaging hair, damaging skin, and/or toxic exposure. Application of the liquid pigment 301 by the non-professional may prolong the time between visits to a hair care professional.

In one or more embodiments, the material of the applicator tip may be substantially saturated with the semi-permanent liquid pigment 301. The applicator may be capable of instantaneous application upon touching the hair without leaking the liquid pigment 301. The liquid pigment 301 may be capable of being instantly deposited on the hair and may allow for increased coverage upon each application. The liquid pigment 301 may be applied to cover the hair shaft evenly.

The example below shows the ingredients of a black liquid pigment 301, in accordance with one embodiment. All concentrations are by percent weight unless otherwise indicated.

| INCI Name | CAS Registry # | % Weight |
|---|---|---|
| Water | 7732-18-5 | To: 100.0 |
| Polyacrylic Acid | 9003-01-4 | 0.4 |
| Acrylates/Octylacrylamide Copolymer | 129702-02-9 | 8.9 |
| Triethanolamine | 102-71-6 | 1.3 |
| Sorbitan Sesquioleate | 8007-43-0 | 2.1 |
| Propylene Glycol | 57-55-6 | 7.3 |
| Polyvinylpyrrolidone | 9003-39-8 | 9.5 |
| Black Oxide of Iron (CI: 77499) | 12227-89-3 | 1.5-2.4 |

The example below shows the ingredients of a brown liquid pigment 301, in accordance with one embodiment. All concentrations are by percent weight unless otherwise indicated.

| INCI Name | CAS Registry # | % Weight |
|---|---|---|
| Water | 7732-18-5 | To: 100.0 |
| Polyacrylic Acid | 9003-01-4 | 0.4 |
| Acrylates/Octylacrylamide Copolymer | 129702-02-9 | 8.9 |
| Triethanolamine | 102-71-6 | 1.3 |
| Sorbitan Sesquioleate | 8007-43-0 | 2.1 |
| Propylene Glycol | 57-55-6 | 7.3 |
| Polyvinylpyrrolidone | 9003-39-8 | 9.5 |
| Yellow Oxide of Iron (CI: 77492) | 1345-27-3 | 12.5 |

The example below shows the ingredients of a light golden brown liquid pigment 301, in accordance with one embodiment. All concentrations are by percent weight unless otherwise indicated.

| INCI Name | CAS Registry # | % Weight |
|---|---|---|
| Water | 7732-18-5 | To: 100.0 |
| Polyacrylic Acid | 9003-01-4 | 0.4 |
| Acrylates/Octylacrylamide Copolymer | 129702-02-9 | 8.9 |
| Triethanolamine | 102-71-6 | 1.3 |
| Sorbitan Sesquioleate | 8007-43-0 | 2.1 |
| Propylene Glycol | 57-55-6 | 7.3 |
| Polyvinylpyrrolidone | 9003-39-8 | 9.5 |
| Phenoxyethanol | 122-99-6 | 0.6 |
| Yellow Oxide of Iron (CI: 77492) | 64294-91-3 | 8.0 |
| Brown Oxide of Iron (CI: 77491) | 52357-70-7 | 7.0 |

In one or more embodiments, the method may further comprise the use of a comb to aid in the application of the liquid pigment 301 to hair roots while avoiding application of the hair dye to the underlying skin. The comb should be thin and small enough to allow easy access of the hair roots, thereby enabling application of the liquid pigment 301 to the very root of the hair, while shielding the scalp, temple, or other skin surface form misapplication of the liquid pigment 301. The comb may expose the surface of hair roots and the applicator may be run along the comb to cover the hair root at the base.

In one or more embodiments, the method comprises a precautionary patch test on an area of skin at least 48 hours prior to application of the liquid pigment 301 to ensure the liquid pigment 301 does not elicit an allergic response and/or skin irritation. However, the general mildness of the liquid pigment 301 as previously described should allow application of the liquid pigment 301 to areas close to the skin such as hair roots.

In one or more embodiments, the method may comprise applying the liquid pigment 301 to eyebrows, eyelashes, mustache, and/or beard. The liquid pigment 301 may also be applied to highlight hair, color overgrown hair roots, change a dominant hair color, and/or maintain a hair color or style between visits to a hair care professional. Overgrown hair roots may be gray, blond, or any color different from the preferred hair color.

In one or more embodiments, the method may further involve removing the liquid pigment 301 from the hair surface by one or more washings with detergent. The acidity of the liquid pigment 301 may determine the degree of permanence of the liquid pigment 301.

In one aspect, the present embodiment provides a hair coloring kit comprising the liquid pigment 301 disclosed earlier and one or more components for the localized application of the liquid pigment 301. The following figures provide examples of components that may be used to apply the liquid pigment 301 according the above methods and that may be included in the hair coloring kit.

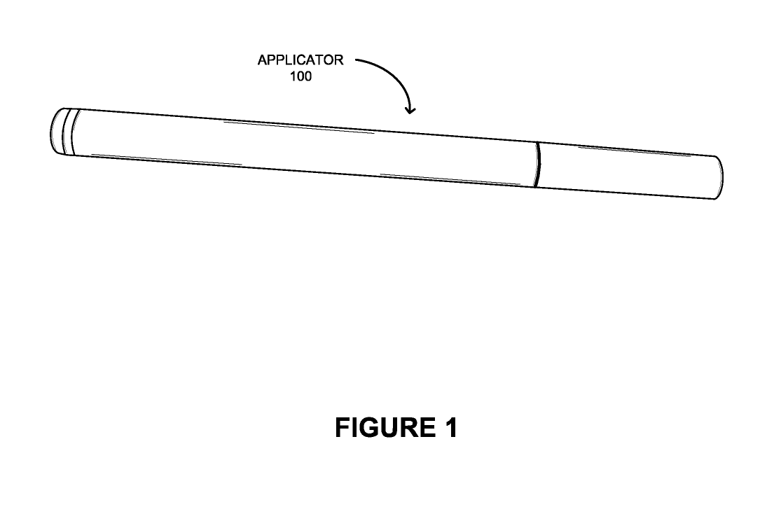

FIG. 1 is a perspective view of an applicator 100 configured to apply the liquid pigment 301. The applicator 100 may be cylindrical, may approximate a cylindrical shape, and/or may approximate a pen-like shape. Further, the hair coloring device may be of a transparent or opaque material that allows the liquid pigment 301 enclosed to be readily visible.

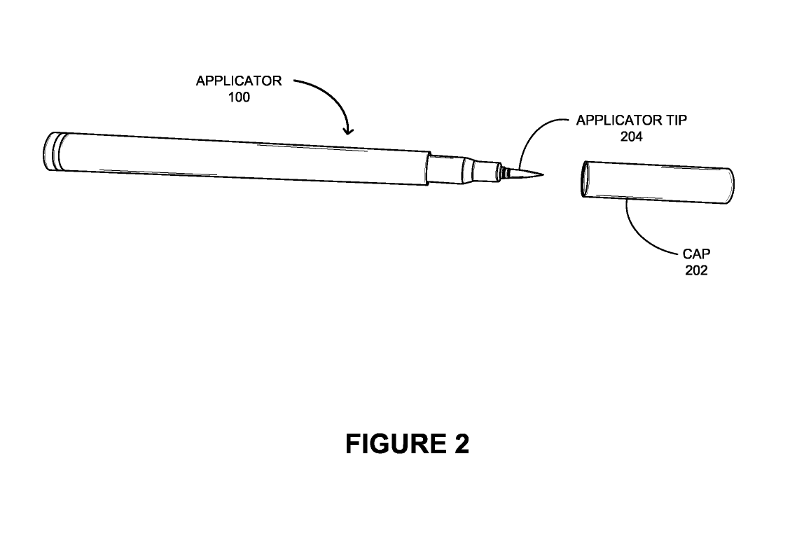

FIG. 2 is a perspective view of the applicator 100 of FIG. 1 with a cap 202 removed displaying an applicator tip 204. The applicator tip 204 may be fiber, felt, sponge and/or another material commonly understood by one of ordinary skill in the art to be able to wick and apply a liquid pigment 301. The applicator tip should allow for the flow of the liquid pigment 301 without clogging. The applicator tip may be removable and/or exchangeable. The cap 202 may form an airtight seal around the applicator tip 204 to prevent drying out of the applicator tip 204.

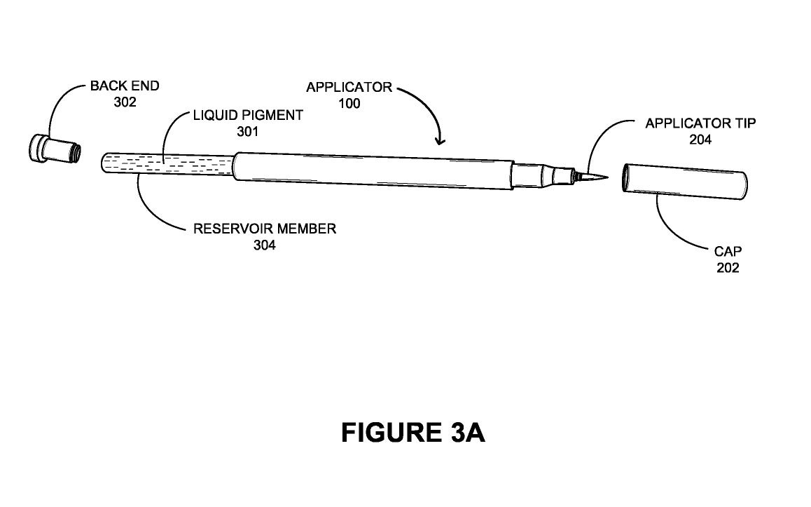

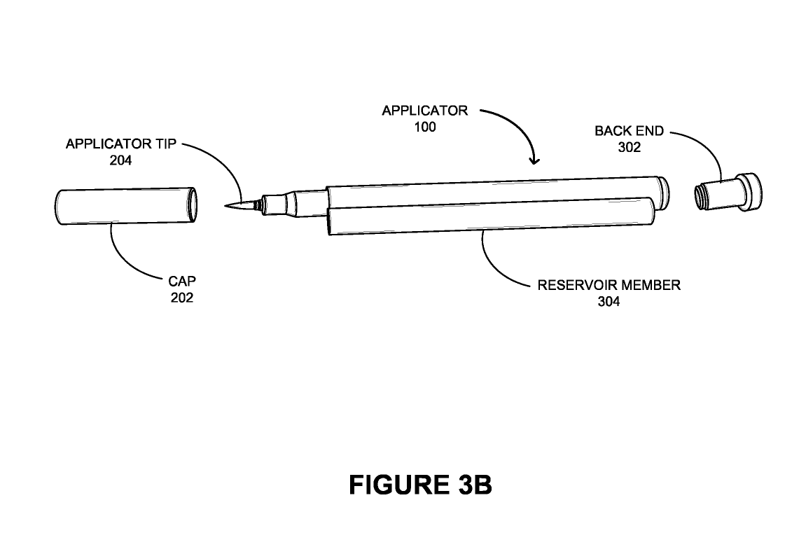

FIG. 3A is a perspective view of the applicator 100 of FIG. 1 with a back end 302 removed displaying a reservoir member 304 partially removed. The reservoir member 304 may house one or more liquid pigment 301s. The reservoir member 304 may be of a transparent or opaque material that allows the color of a liquid pigment 301 in the reservoir member 304 to be readily observed. The reservoir member 304 may be designed for single use or for reuse by replenishing of the liquid pigment 301. When the hair coloring device is fully assembled, the reservoir member 304 may communicate with an applicator tip 204 through overlapping fibers and/or another means of communication familiar to one of ordinary skill in the art. The applicator tip 204 may wick (e.g. by capillary action) or otherwise transfer the liquid pigment 301 from the reservoir member 304. A cap 202 may provide an airtight seal that prevents drying out of the applicator tip 204 and/or prevents depletion of the liquid pigment 301 in the reservoir member 304.

FIG. 3B is a perspective view of the applicator 100 of FIG. 1 with the reservoir member 304 fully removed. The reservoir member 304 may be fully removable from the body of the hair coloring device and may be exchangeable with other reservoir members. The other reservoir members may contain different liquid pigment 301s.

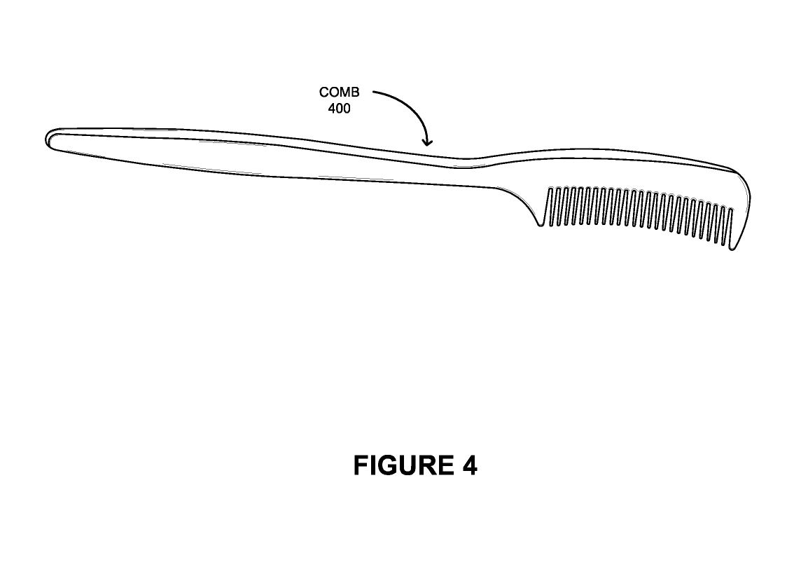

FIG. 4 is a perspective view of a comb 400 configured to aid in the application of a liquid pigment 301. In one or more embodiments, the comb 400 may be used to separate and/or expose hair surfaces to be colored. In particular the comb 400 may be used to present the base of hair roots to the applicator 100 of FIG. 1 while shielding underlying skin from the applicator 100. The applicator 100 may then be run along the comb 400 to apply the liquid pigment 301 to the base of the hair roots. The comb 400 should be thin to allow for the very base of the hair roots to be exposed while still shielding the underlying skin during this process. Further, the comb 400 should be small enough to allow ease of access to localized regions of hair.

In one or more embodiments, the hair coloring kit may provide a reservoir member preloaded with the liquid pigment 301. Further, the hair coloring kit may provide an applicator pre-configured to house the pre-loaded reservoir. Alternatively, the kit may provide the liquid pigment 301 in a form that can be used to load and/or refill the reservoir member.

In one or more embodiments, the hair coloring kit may provide a selection of liquid pigment 301s. The liquid pigment 301s in the selection may be of different degrees of permanence and/or different colors. Further, the liquid pigment 301s may be in a form that could be used to refill a reusable reservoir member. Alternatively, each liquid pigment 301 in the selection may be pre-loaded in a separate reservoir member.

In one or more embodiments, the hair coloring kit may comprise one or more reservoir members and/or applicators in addition to one or more liquid pigment 301s. The hair coloring kit may also include one or more combs. The combs may be thin enough to allow for application of the liquid pigment 301 to the root of the hair while still protecting the underlying skin. The hair coloring kit may further comprise other hair cosmetic products that would aid in the application of the liquid pigment 301 and would be familiar to one of ordinary skill in the art.

In one or more embodiments, when the hair coloring kit comprises multiple applicators in addition to one or more liquid pigment 301s, the applicators may be of different structure and/or function. For example, applicators may differ in the size, shape, and/or material of the applicator tip. Further, the applicators may differ in the communication between the reservoir member and the applicator tip, resulting in different flow rates of the liquid pigment 301. Still further, the applicators may differ in their exterior appearance, such as shape and color. In a further example, the color of the applicator may be matched to the color of the liquid pigment 301 housed therein.

In one or more embodiments, when the hair coloring kit comprises multiple combs in addition to one or more liquid pigment 301s, the combs may be of different structure and/or function. For example, combs may differ in width, fineness of teeth, and/or overall size. Different combs may be useful for different areas, applications and/or types of hair.

In one aspect, a hair coloring kit includes an applicator pen to apply a dye composition to a hair surface through a capillary action through which the dye composition is extruded through the applicator pen onto the hair surface. The dye composition is removable from a skin adjacent to the hair surface when washed using a detergent material. The dye composition includes an inorganic pigment which further includes an iron oxide in a concentration between 1.5% weights to 12.5% weight and is substantially free of ethyl alcohol. A viscosity of the dye composition is less than 100 cP to prevent clogging of a chamber of the applicator pen and a comb that is usable with the applicator pen to expose a hair root while shielding a skin that underlies the hair surface.

The hair coloring kit may further include a reservoir member in a central chamber of the applicator pen that stores the dye composition. The reservoir member is removable from the applicator pen when a back-end assembly is unfastened from an end opposite to an applicator tip of the applicator pen to expose the reservoir member. This may permit the reservoir member to slide outward from a cavity of the central chamber of the applicator pen in the end opposite to the applicator tip.

The hair coloring kit may include a removable cap to secure the applicator tip of the applicator pen and to prevent the dye composition from drying and escaping from the applicator pen when the removable cap is secured to the applicator pen. The dye composition may automatically extrude from the applicator tip of the applicator pen at a rate of volume that does not create an excess leakage of the dye composition onto a surrounding skin. The dye composition extruding from the applicator tip may be substantially saturated in the hair surface based on a regulated rate of extrusion of the dye composition.

The wicking action of the applicator pen may permit the dye composition from extruding from the applicator pen through the applicator tip onto the hair surface.

The dye composition may include water, a water soluble film forming polymer, a surfactant, and/or an inorganic pigment.

The water soluble film forming polymer may be a polyacrylic acid, an acrylate/octylacrylamide copolymer, and/or a polyvinylpyrrolidone polymer.

A body of the applicator pen may be a transparent visibility and/or an opaque visibility to permit a level of the dye composition to be visually observable to a user of the applicator pen.

In another aspect, a hair coloring kit includes an applicator pen to apply a dye composition to a hair surface through a capillary action through which the dye composition is extruded through the applicator pen onto the hair surface. The dye composition is removable from a skin adjacent to the hair surface when washed using a detergent material. The dye composition includes an inorganic pigment which further includes an iron oxide in a concentration between 1.5% weights to 12.5% weight and is substantially free of ethyl alcohol. A viscosity of the dye composition is less than 100 cP to prevent clogging of a chamber of the applicator pen.

In yet another aspect, a method includes a dye composition to a hair surface through a capillary action through which the dye composition is extruded through an applicator pen onto the hair surface. The dye composition is removable from a skin adjacent to the hair surface when washed using a detergent material. The dye composition includes an inorganic pigment which further includes an iron oxide in a concentration between 1.5% weights to 12.5% weight and is substantially free of ethyl alcohol. A viscosity of the dye composition is less than 100 cP to prevent clogging of a chamber of the applicator pen. The method also exposes a hair root while shielding a skin that underlies the hair surface using a comb utilized in conjunction with the applicator pen.

In addition, changing or enhancing hair color has been a notably time-consuming, difficult, sloppy and toxic process, even when performed by a hair professional. Hair coloring by a non-professional user is usually even more time-consuming, difficult, sloppy and toxic than when done by the hair professional. Thus, the hair coloring is even more frustrating for the nonprofessional when done at home than when done by the hair professional. Much of the reason for large time-consumption, difficulty, sloppiness and toxicity of the hair coloring process has been due to hair dye compositions and methods available for hair coloring.

Typically, hair coloring is time-consuming because permanent and demi-permanent hair dyes compositions must penetrate the hair shaft in order to effectively color the hair. Penetration of the hair shaft by permanent and demi-permanent hair dye compositions can take more than an hour, making it impossible to color hair on short notice.

Also, due to the constant growth of hair, an individual is often presented with a choice of having gray roots or visiting the hair professional on a weekly basis. This time investment is a significant problem for an individual that desires to color his or her own hair. For example, when an individual identifies gray hair roots immediately before an important social occasion, the individual does not have time to dye the gray hair roots with permanent or demi-permanent hair dye. In addition, permanent and demi-permanent hair dyes often comprise ethyl alcohol as a solvent, which has a drying effect on hair.

Conventional methods of hair coloring are also difficult for an individual because complete and even coverage with the hair dye composition may be difficult to achieve. Moreover, in order to color gray or overgrown roots, hair dye composition must be applied to the root of the hair where the hair is parted. Further, the hair dye composition must be distributed along the scalp over the entire part in the hair in order to reach all of the hair roots. This procedure must be repeated until all of the roots have even coverage with the hair dye composition.

Conventional methods of applying hair dye composition include squeeze tubes with various sizes of openings or use of brushes which are similar to small paint brushes. Both methods can result in a messy application. When squeeze tubes are used to apply the hair dye composition to the scalp and root of the hair, a user (which hereinafter may refer either to a layperson or a professional) must use a gloved hand in order to ensure that the hair coloring is distributed evenly down each part of the hair. Since permanent and demi-permanent hair dye compositions can permanently stain clothes, skin and other surfaces, the user must be extremely careful both until and even after the soiled glove is removed. Once the glove is removed, it must be turned inside-out to avoid contaminating surrounding surfaces. Since hair coloring with permanent and demi-permanent hair dye compositions involves a number of intermediate steps and may require a time span in excess of an hour, the gloved hand that is contaminated with hair dye during the time span may prove to be a great inconvenience to the user. The continual need for gloving and re-gloving in order to prevent contact of the skin and surrounding area with the hair dye may also be inconvenient to the user.

If a paintbrush is used to apply the hair dye composition to the scalp at the root, complete coverage is not always achieved, and further distribution with the gloved hand may be necessary to ensure complete and even coverage. It is apparent, then, that the problems one may encounter with this method of application are similar to those previously mentioned including time consumption, difficulty and sloppiness. Additionally, hair coloring is typically achieved through the use of toxic chemicals including oxidizing agents and couplers (e.g. aniline derivatives) in an alkaline (i.e. high pH, basic) environment. Typically, oxidizing agents include hydrogen peroxide and the alkaline environment is provided by ammonia. Use of these chemicals can result in a range of adverse effects, including temporary skin irritation and allergy, hair breakage, skin discoloration and unexpected hair color results. The temporary skin irritation typically associated with hair coloring may discourage the user from coloring hair near the skin, such as hair roots, eyebrows, mustache, and beard.

In another embodiment, a hair coloring kit comprising a liquid pigment 301 having a first property that enables the liquid pigment 301 to dry upon contact with a hair surface, and which exhibits a second property through which the liquid pigment 301 is removable from the hair surface upon a first wash with a shampoo material, and an applicator pen to apply the liquid pigment 301 to the hair surface through a capillary action through which the liquid pigment 301 is extruded through the applicator pen onto the hair surface. The liquid pigment 301 is removable from a skin adjacent to the hair surface when washed using the shampoo material. The liquid pigment 301 includes an inorganic pigment comprising at least one iron oxide in a concentration between 1.5% weight to 12.5% weight. The liquid pigment 301 is substantially free of ethyl alcohol. A viscosity of the liquid pigment 301 is less than 100 centipoise (cP) to prevent clogging of a chamber of the applicator pen. The hair surface is optionally treatable with a dye composition while the liquid pigment 301 is on the hair surface and without requiring a wax removal process. Further, a comb is usable with the applicator pen to expose a hair root while shielding a skin that underlies the hair surface.

The hair coloring kit may include a reservoir member in a central chamber of the applicator pen that stores the liquid pigment 301. The reservoir member may be removable from the applicator pen when a back-end assembly is unfastened from an end opposite to an applicator tip of the applicator pen such that the back-end assembly to expose the reservoir member and to permit the reservoir member to slide outward from a cavity of the central chamber of the applicator pen in the end opposite to the applicator tip.

The hair coloring kit may include a removable cap on secure the applicator tip of the applicator pen and to prevent the liquid pigment 301 from at least one of drying and escaping from the applicator pen when the removable cap is secured to the applicator pen. The liquid pigment 301 may automatically extrude from the applicator tip of the applicator pen at a rate of volume that does not create an excess leakage of the liquid pigment 301 onto a surrounding skin. The liquid pigment 301 extruding from the applicator tip may be substantially saturated in the hair surface based on a regulated rate of extrusion of the liquid pigment 301. A wicking action of the applicator pen may permit the liquid pigment 301 from extruding from the applicator pen through the applicator tip onto the hair surface. The liquid pigment 301 may include a water, a water soluble film forming polymer, a surfactant, and/or an inorganic pigment. The water soluble film forming polymer may a polyacrylic acid, an acrylate/octylacrylamide copolymer, and/or a polyvinylpyrrolidone polymer. A body of the applicator pen may be at least one of a transparent visibility and an opaque visibility to permit a level of the liquid pigment 301 to be visually observable to a user of the applicator pen.

In yet another embodiment, a hair coloring kit includes a liquid pigment 301 having a first property that enables the liquid pigment 301 to dry upon contact with a hair surface, and which exhibits a second property through which the liquid pigment 301 washes out upon a wash with a shampoo material, and an applicator pen to apply the liquid pigment 301 to the hair surface through a capillary action through which the liquid pigment 301 is extruded through the applicator pen onto the hair surface. The liquid pigment 301 is removable from a skin adjacent to the hair surface when washed using a shampoo material. The liquid pigment 301 includes an inorganic pigment comprising at least one iron oxide in a concentration between 1.5% weight to 12.5% weight. The liquid pigment 301 is substantially free of ethyl alcohol. The hair surface is optionally treatable with a dye composition while the liquid pigment 301 is on the hair surface and without requiring a wax removal process. A viscosity of the liquid pigment 301 is less than 100 cP to prevent clogging of a chamber of the applicator pen.

In a further embodiment, a method includes applying a liquid pigment 301 to a hair surface through a capillary action through which the liquid pigment 301 is extruded through an applicator pen onto the hair surface. The liquid pigment 301 is removable from a skin adjacent to the hair surface when washed using a shampoo material. The liquid pigment 301 may include an inorganic pigment comprising at least one iron oxide in a concentration between 1.5% weight to 12.5% weight. The liquid pigment 301 is substantially free of ethyl alcohol. A viscosity of the liquid pigment 301 is less than 100 cP to prevent clogging of a chamber of the applicator pen. The method includes exposing a hair root while shielding a skin that underlies the hair surface using a comb utilized in conjunction with the applicator pen. The hair surface is optionally treatable with a dye composition while the liquid pigment 301 is on the hair surface and without requiring a wax removal process. The liquid pigment 301 has a property that enables the liquid pigment 301 to dry upon contact with the hair surface and remain in liquid form while in the applicator pen.

A number of embodiments have been described. Nevertheless, it will be understood that various modifications may be made without departing from the spirit and scope of the claimed embodiment. In addition, other steps may be provided, or steps may be eliminated, from the described flows, and other components may be added to, or removed from, the described systems. Accordingly, other embodiments are within the scope of the following claims.

What is claimed is:

1. A hair coloring kit comprising:
a liquid pigment having a first property that enables the liquid pigment to dry upon contact with a hair surface, and which exhibits a second property through which the liquid pigment is removable from the hair surface upon a first wash with a shampoo material;
an applicator pen to apply the liquid pigment to the hair surface through a capillary action through which the liquid pigment is extruded through the applicator pen onto the hair surface;
wherein the liquid pigment is removable from a skin adjacent to the hair surface when washed using the shampoo material,
wherein the liquid pigment includes an inorganic pigment comprising at least one iron oxide in a concentration between 1.5% weight to 12.5% weight,
wherein the liquid pigment is substantially free of ethyl alcohol,
wherein a viscosity of the liquid pigment is less than 100 cP to prevent clogging of a chamber of the applicator pen,
wherein the hair surface to be optionally treatable with a dye composition while the liquid pigment is on the hair surface and without requiring a wax removal process, and
a comb that is usable with the applicator pen to expose a hair root while shielding a skin that underlies the hair surface.

2. The hair coloring kit of claim 1 further comprising:
a reservoir member in a central chamber of the applicator pen that stores the liquid pigment,
wherein the reservoir member is removable from the applicator pen when a back-end assembly is unfastened from an end opposite to an applicator tip of the applicator pen such that the back-end assembly to expose the reservoir member and to permit the reservoir member to slide outward from a cavity of the central chamber of the applicator pen in the end opposite to the applicator tip.

3. The hair coloring kit of claim 2 further comprising:
a removable cap on secure the applicator tip of the applicator pen and to prevent the liquid pigment from at least one of drying and escaping from the applicator pen when the removable cap is secured to the applicator pen, and
wherein the liquid pigment to automatically extrude from the applicator tip of the applicator pen at a rate of volume that does not create an excess leakage of the liquid pigment onto a surrounding skin,
wherein the liquid pigment extruding from the applicator tip to be substantially saturated in the hair surface based on a regulated rate of extrusion of the liquid pigment.

4. The hair coloring kit of claim 1 wherein a wicking action of the applicator pen to permit the liquid pigment from extruding from the applicator pen through the applicator tip onto the hair surface.

5. The hair coloring kit of claim 1 wherein the liquid pigment comprises a water, a water soluble film forming polymer, a surfactant, and an inorganic pigment.

6. The hair coloring kit of claim 5 wherein the water soluble film forming polymer is at least one of a polyacrylic acid, an acrylate/octylacrylamide copolymer, a polyvinylpyrrolidone polymer.

7. The hair coloring kit of claim 1:
wherein a body of the applicator pen is at least one of a transparent visibility and an opaque visibility to permit a level of the liquid pigment to be visually observable to a user of the applicator pen.

8. A hair coloring kit comprising:
a liquid pigment having a first property that enables the liquid pigment to dry upon contact with a hair surface, and which exhibits a second property through which the liquid pigment washes out upon a wash with a shampoo material;
an applicator pen to apply the liquid pigment to the hair surface through a capillary action through which the liquid pigment is extruded through the applicator pen onto the hair surface;
wherein the liquid pigment is removable from a skin adjacent to the hair surface when washed using a shampoo material,
wherein the liquid pigment includes an inorganic pigment comprising at least one iron oxide in a concentration between 1.5% weight to 12.5% weight,
wherein the liquid pigment is substantially free of ethyl alcohol,
wherein the hair surface to be optionally treatable with a dye composition while the liquid pigment is on the hair surface and without requiring a wax removal process, and
wherein a viscosity of the liquid pigment is less than 100 cP to prevent clogging of a chamber of the applicator pen.

9. The hair coloring kit of claim 8 further comprising:
a comb that is usable with the applicator pen to expose a hair root while shielding a skin that underlies the hair surface;
a reservoir member in a central chamber of the applicator pen that stores the liquid pigment,
wherein the reservoir member is removable from the applicator pen when a back-end assembly is unfastened from an end opposite to an applicator tip of the applicator pen such that the back-end assembly to expose the reservoir member and to permit the reservoir member to slide outward from a cavity of the central chamber of the applicator pen in the end opposite to the applicator tip.

10. The hair coloring kit of claim 9 further comprising:
a removable cap on secure the applicator tip of the applicator pen and to prevent the liquid pigment from at least one of drying and escaping from the applicator pen when the removable cap is secured to the applicator pen, and
wherein the liquid pigment to automatically extrude from the applicator tip of the applicator pen at a rate of volume that does not create an excess leakage of the liquid pigment onto a surrounding skin,
wherein the liquid pigment extruding from the applicator tip to be substantially saturated in the hair surface based on a regulated rate of extrusion of the liquid pigment.

11. The hair coloring kit of claim 8 wherein a wicking action of the applicator pen to permit the liquid pigment from extruding from the applicator pen through the applicator tip onto the hair surface.

12. The hair coloring kit of claim 8 wherein the liquid pigment comprises a water, a water soluble film forming polymer, a surfactant, and an inorganic pigment.

13. The hair coloring kit of claim 12 wherein the water soluble film forming polymer is at least one of a polyacrylic acid, an acrylate/octylacrylamide copolymer, a polyvinylpyrrolidone polymer.

14. The hair coloring kit of claim 8
wherein a body of the applicator pen is at least one of a transparent visibility and an opaque visibility to permit a level of the liquid pigment to be visually observable to a user of the applicator pen.

15. A method comprising:
applying a liquid pigment to a hair surface through a capillary action through which the liquid pigment is extruded through an applicator pen onto the hair surface;
wherein the liquid pigment is removable from a skin adjacent to the hair surface when washed using a shampoo material,
wherein the liquid pigment includes an inorganic pigment comprising at least one iron oxide in a concentration between 1.5% weight to 12.5% weight,
wherein the liquid pigment is substantially free of ethyl alcohol, and
wherein a viscosity of the liquid pigment is less than 100 cP to prevent clogging of a chamber of the applicator pen; and
exposing a hair root while shielding a skin that underlies the hair surface using a comb utilized in conjunction with the applicator pen,
wherein the hair surface to be optionally treatable with a dye composition while the liquid pigment is on the hair surface and without requiring a wax removal process, and
wherein the liquid pigment having a property that enables the liquid pigment to dry upon contact with the hair surface and remain in liquid form while in the applicator pen.

16. The method of claim 15:
wherein a reservoir member in a central chamber of the applicator pen that stores the liquid pigment, and
wherein the reservoir member is removable from the applicator pen when a back-end assembly is unfastened from an end opposite to an applicator tip of the applicator pen such that the back-end assembly to expose the reservoir member and to permit the reservoir member to slide outward from a cavity of the central chamber of the applicator pen in the end opposite to the applicator tip.

17. The method of claim 16:
wherein a removable cap secures the applicator tip of the applicator pen and prevents the liquid pigment from at least one of drying and escaping from the applicator pen when the removable cap is secured to the applicator pen, and
wherein the liquid pigment automatically extrudes from the applicator tip of the applicator pen at a rate of volume that does not create an excess leakage of the liquid pigment onto a surrounding skin,
wherein the liquid pigment extruding from the applicator tip to be substantially saturated in the hair surface based on a regulated rate of extrusion of the liquid pigment.

18. The method of claim 15 wherein a wicking action of the applicator pen permits the liquid pigment from extruding from the applicator pen through the applicator tip onto the hair surface.

19. The method of claim 15 wherein the liquid pigment comprises a water, a water soluble film forming polymer, a surfactant, and an inorganic pigment.

20. The method of claim 19:
wherein the water soluble film forming polymer is at least one of a polyacrylic acid, an acrylate/octylacrylamide copolymer, a polyvinylpyrrolidone polymer.
wherein a body of the applicator pen is at least one of a transparent visibility and an opaque visibility to permit a level of the liquid pigment to be visually observable to a user of the applicator pen.
